(19) United States
(12) Patent Application Publication
(10) Pub. No.: US 2025/0205454 A1
(43) Pub. Date: Jun. 26, 2025

(54) SECUREMENT DEVICE FOR SECURING MEDICAL ARTICLE, KIT COMPRISING SECUREMENT DEVICE, AND METHOD OF USE THEREOF

(71) Applicant: Solventum Intellectual Properties Company, Maplewood, MN (US)

(72) Inventors: Brian E. SPIEWAK, Inver Grove Heights, MN (US); Del R. LAWSON, Cottage Grove, MN (US); Dylan T. COSGROVE, Oakdale, MN (US); Abigail M. FROECHTENIGT, Maple Grove, MN (US); Joseph D. PREMKUMAR, Minneapolis, MN (US); Zhaojin LYU, Chengdu, Sichuan (CN)

(21) Appl. No.: 18/850,791

(22) PCT Filed: Mar. 21, 2023
(86) PCT No.: PCT/IB2023/052762

§ 371 Date: Sep. 25, 2024

Related U.S. Application Data
(60) Provisional application No. 63/324,998, filed on Mar. 29, 2022.

(51) Int. Cl. A61M 25/02

(52) U.S. Cl. CPC A61M 25/02; A61M 2025/024; A61M 2025/0273; A61M 2025/0286

(57) ABSTRACT

A securement device includes a main body including a first major surface, a second major surface opposite to the first major surface, a perimeter extending between the first and second major surfaces, and a slot extending from the perimeter and extending through the main body from the first major surface to the second major surface. The securement device further includes a clip extending from the first major surface and a plurality of microhooks extending from the second major surface. Each microhook includes a base disposed on the second major surface and a tip distal to the base. The plurality of microhooks includes at least one microhook and at least one opposing microhook facing the at least one microhook, such that the respective tips of the at least one microhook and the at least one opposing microhook point towards each other.

### TECHNICAL FIELD

The present disclosure relates to a securement device for securing a medical article, a kit including the securement device, and a method of using the kit.

### BACKGROUND

Various medical treatments often require use of medical articles (e.g., catheters) having medical lines. Such medical articles may be used for various purposes, such as feeding, air supply, liquid removal, and/or to administer medications and fluids to a patient. In many instances, the medical articles may need to be secured to the patient. Securing the medical articles to the patient may facilitate proper positioning of the medical articles, which may further prevent leaks or interruptions in medication dosing, minimize patient discomfort, and limit tangling, catching, and/or dislodging of the medical lines due to a movement of the patient.

One common way of securing the medical articles to the patient is by taping the medical articles (e.g., by a surgical tape) to a skin of the patient. However, taping may be time consuming and labor intensive. Tapes may also collect contaminants, and therefore may need to be frequently replaced. In addition, taping may not be effective in securing the medical articles to the patient, may cause undesired motion of the medical articles relative to the skin, and may cause adhesive-related skin injuries to the patient.

### SUMMARY

In a first aspect, the present disclosure provides a securement device for securing a medical article having at least one medical line insertable into a skin of a user at an insertion site. The securement device includes a main body including a first major surface. The main body further includes a second major surface opposite to the first major surface. The second major surface is configured to face the skin of the user. The main body further includes a perimeter extending between the first major surface and the second major surface. The main body further includes a slot extending from the perimeter and extending through the main body from the first major surface to the second major surface. The slot is configured to at least partially and slidably receive the at least one medical line therethrough. The securement device further includes a clip coupled to the main body and extending from the first major surface. The clip is configured to at least partially receive the medical article therethrough and removably secure the medical article to the main body. The securement device further includes a plurality of microhooks coupled to the main body and extending from the second major surface. The plurality of microhooks are spaced apart from each other and configured to be at least partially inserted into the skin of the user to secure the main body to the skin. Each microhook from the plurality of microhooks includes a base disposed on the second major surface and a tip distal to the base. The plurality of microhooks includes at least one microhook and at least one opposing microhook facing the at least one microhook, such that the respective tips of the at least one microhook and the at least one opposing microhook point towards each other.

In a second aspect, the present disclosure provides a kit. The kit includes a securement device for securing a medical article having at least one medical line insertable into a skin of a user at an insertion site. The securement device includes a main body including a first major surface. The main body further includes a second major surface opposite to the first major surface. The second major surface is configured to face the skin of the user. The main body further includes a perimeter extending between the first major surface and the second major surface. The main body further includes a slot extending from the perimeter and extending through the main body from the first major surface to the second major surface. The slot is configured to at least partially and slidably receive the at least one medical line therethrough. The securement device further includes a clip coupled to the main body and extending from the first major surface. The clip is configured to at least partially receive the medical article therethrough and removably secure the medical article to the main body. The securement device further includes a plurality of microhooks coupled to the main body and extending from the second major surface. The plurality of microhooks are spaced apart from each other and configured to be at least partially inserted into the skin of the user to secure the main body to the skin. Each microhook from the plurality of microhooks includes a base disposed on the second major surface and a tip distal to the base. The plurality of microhooks includes at least one microhook and at least one opposing microhook facing the at least one microhook, such that the respective tips of the at least one microhook and the at least one opposing microhook point towards each other. The kit further includes a gel pad configured to be at least partially received within the aperture and to cover the insertion site. The gel pad includes chlorhexidine gluconate.

In a third aspect, the present disclosure provides a method of using the kit of the second aspect. The method includes positioning the securement device relative to the skin of the user, such that the second major surface of the main body faces the skin. The method further includes slidably inserting the at least one medical line through the slot, such that the at least one medical line is at least partially received through the aperture of the main body. The method further includes inserting the medical article at least partially through the clip to removably secure the medical article to the main body. The method further includes inserting the plurality of microhooks into the skin of the user, such that the main body is secured to the skin of the user. The method further includes inserting the gel pad at least partially within the aperture, such that the gel pad covers the insertion site.

### BRIEF DESCRIPTION OF THE DRAWINGS

Exemplary embodiments disclosed herein may be more completely understood in consideration of the following detailed description in connection with the following figures. The figures are not necessarily drawn to scale. In particular, thicknesses of certain layers in proportion to certain other items are exaggerated for ease of illustration and clarity purposes. Like numbers used in the figures refer to like components. However, it will be understood that the use of a number to refer to a component in a given figure is not intended to limit the component in another figure labeled with the same number.

FIG. 1 is a schematic top perspective view of a securement device according to an embodiment of the present disclosure;

FIG. 2 is a schematic bottom perspective view of the securement device of FIG. 1 according to an embodiment of the present disclosure;

FIG. 3 is a schematic bottom view of the securement device of FIG. 1 according to an embodiment of the present disclosure;

FIG. 4A is a schematic top perspective view of a microhook of the securement device of FIG. 1 according to an embodiment of the present disclosure;

FIG. 4B is a schematic bottom perspective view of the microhook of FIG. 4A according to an embodiment of the present disclosure;

FIG. 4C is a schematic side view of the microhook of FIG. 4A according to an embodiment of the present disclosure;

FIG. 5 is a schematic top perspective view of a portion of the securement device of FIG. 1 according to an embodiment of the present disclosure;

FIG. 6 is a schematic block diagram of a kit according to an embodiment of the present disclosure;

FIG. 7 is a flowchart depicting various steps of a method of using the kit of FIG. 6 according to an embodiment of the present disclosure; and

FIGS. 8A-8I schematically illustrate the method of FIG. 7.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials includes less than about 10 weight % of each of the first and second materials.

As used herein, “at least one of A and B” should be understood to mean “only A, only B, or both A and B”.

Unless specified or limited otherwise, the terms “attached,” “connected,” “coupled,” and variations thereof, are used broadly and encompass both direct and indirect attachments, connections, and couplings.

As used herein, the term “configured to” and like is at least as restrictive as the term “adapted to” and requires actual design intention to perform the specified function rather than mere physical capability of performing such a function.

As used herein, the term “spaced apart” refers to elements that are disposed at a distance from one another.

The present disclosure relates to a securement device for securing a medical article having at least one medical line insertable into a skin of a user at an insertion site. The securement device includes a main body including a first major surface. The main body further includes a second major surface opposite to the first major surface. The second major surface is configured to face the skin of the user. The main body further includes a perimeter extending between the first major surface and the second major surface. The main body further includes a slot extending from the perimeter and extending through the main body from the first major surface to the second major surface. The slot is configured to at least partially and slidably receive the at least one medical line therethrough. The securement device further includes a clip coupled to the main body and extending from the first major surface. The clip is configured to at least partially receive the medical article therethrough and removably secure the medical article to the main body. The securement device further includes a plurality of microhooks coupled to the main body and extending from the second major surface. The plurality of microhooks are spaced apart from each other and configured to be at least partially inserted into the skin of the user to secure the main body to the skin. Each microhook from the plurality of microhooks includes a base disposed on the second major surface and a tip distal to the base. The plurality of microhooks includes at least one microhook and at least one opposing microhook facing the at least one microhook, such that the respective tips of the at least one microhook and the at least one opposing microhook point towards each other.

The securement device of the present disclosure may secure the medical article to the skin of the user via the plurality of microhooks. Specifically, the medical article may be secured to the main body by the clip, and the main body may be secured to the skin of the user by the plurality of microhooks. The plurality of microhooks may not penetrate a dermis layer of the skin of the user. Therefore, the securement device may not cause pain and discomfort to the user during use.

The securement device may provide various advantages as compared to conventional securement methods, such as taping. The securement device may be convenient to use and may not need to be replaced periodically. Further, the securement device may be easily applied at a variety of locations on the user. Due to securement via the plurality of microhooks, the securement device may further prevent adhesive-related skin injuries to the user that may arise due to taping.

Since the respective tips of the at least one microhook and the at least one opposing microhook point towards each other, the at least one microhook and the at least one opposing microhook may be securely inserted into the skin of the user, thereby substantially preventing any relative movement between the main body and the skin. Further, the relative configuration of the at least one microhook and the at least one opposing microhook may prevent any accidental detachment of the main body from the skin. Therefore, the securement device may firmly secure the medical article to the skin of the user, thereby preventing dislodgement and accidental removal of the medical article from the skin of the user. As a result, the securement device may prevent various complications such as phlebitis, extravasation/infiltration, leakage, occlusion, and bloodstream infections that may otherwise occur due to movement of the medical article.

In some cases, a gel pad may be used with the securement device to at least partially cover the insertion site. The gel pad may protect the insertion site from micro-organisms and prevent infections.

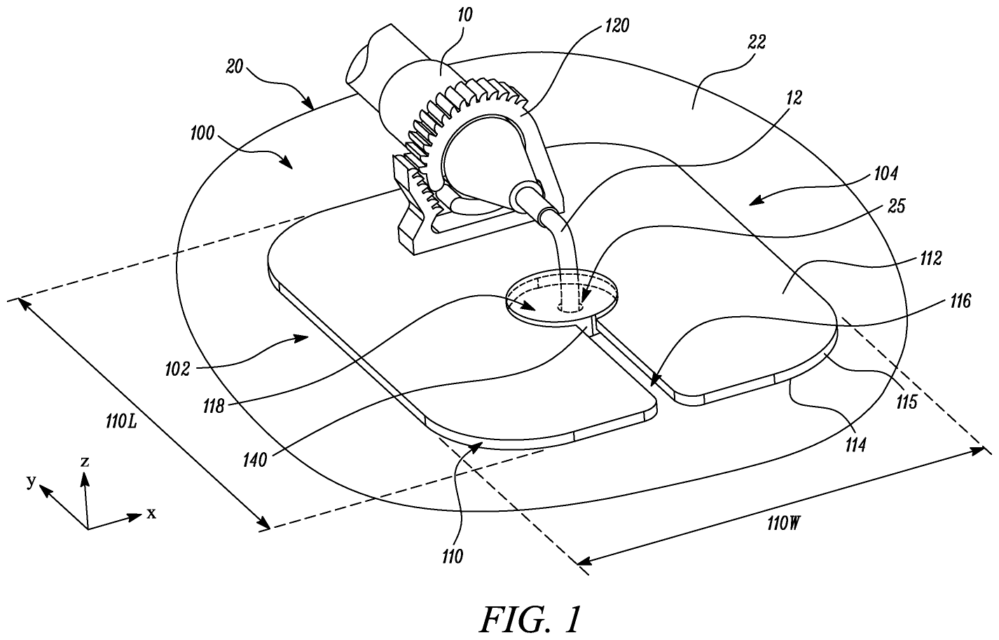

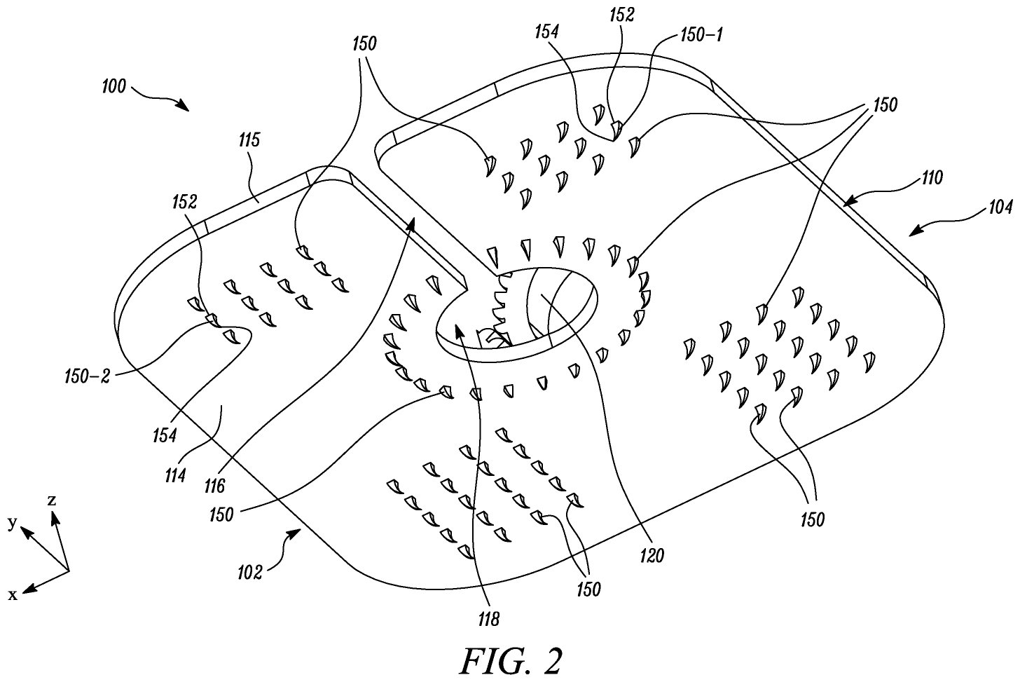

Referring now to the Figures, FIGS. 1 and 2 illustrate a securement device 100 according to an embodiment of the present disclosure. Specifically, FIG. 1 illustrates a schematic top perspective view of the securement device 100 for securing a medical article 10 having at least one medical line 12 insertable into a skin 22 of a user 20 at an insertion site 25. The medical article 10 is partially shown in FIG. 1. Further, FIG. 2 illustrates a bottom perspective view of the securement device 100.

The securement device 100 defines mutually orthogonal x, y, and z-axes. The x-axis is defined along a width of the securement device 100, while the y-axis is defined along a length of the securement device 100. The z-axis is defined along a thickness of the securement device 100.

The securement device 100 includes a main body 110. The main body 110 includes a first major surface 112 and a second major surface 114 opposite to the first major surface 112. The second major surface 114 is configured to face the skin 22 of the user 20. The main body 110 may further include a first end 102 and a second end 104 opposing the first end 102. The first end 102 and the second end 104 may correspond to longitudinal ends of the main body 110 disposed along the y-axis.

The main body 110 further includes a perimeter 115 extending between the first major surface 112 and the second major surface 114. The main body 110 further includes a slot 116 extending from the perimeter 115 and extending through the main body 110 from the first major surface 112 to the second major surface 114. In other words, the main body 110 may define the slot 116 therethrough extending from the perimeter 115. The slot 116 is configured to at least partially and slidably receive the at least one medical line 12 therethrough. The securement device 100 may be slid relative to the at least one medical line 12, such that the slot 116 slidably receives the at least one medical line 12 therethrough.

Upon application of the securement device 100 on the skin 22 of the user 20, a portion of the slot 116 may be aligned with the insertion site 25, such that the at least one medical line 12 is inserted into the skin 22 of the user 20 through the slot 116. Alternatively, as shown in FIG. 1, the main body 110 may further include an aperture 118 extending therethrough and communicating with the slot 116. The aperture 118 may be configured to at least partially receive the at least one medical line 12 therethrough. The securement device 100 may be slid relative to the at least one medical line 12, such that the at least one medical line 12 is at least partially received by the aperture 118 via the slot 116.

In the illustrated embodiment of FIG. 1, the slot 116 is a substantially linear slot extending from the perimeter 115 to the aperture 118. Further, the slot 116 is substantially normal to the perimeter 115. Specifically, the slot 116 is substantially parallel to the y-axis. However, in some other embodiments, the slot 116 may be inclined obliquely to the y-axis. Moreover, in alternative embodiments, the slot 116 may have a curvilinear shape.

As shown in FIG. 1, the aperture 118 may be further configured to at least partially receive a gel pad 140 therein. The gel pad 140 is not shown in FIG. 2 for the purposes of clarity. The gel pad 140 may be configured to be at least partially received within the aperture 118 and to cover the insertion site 25. The gel pad 140 may include any suitable disinfectant and/or antiseptic agent, such as iodopovidone, cetrimide, and the like. In some examples, the gel pad 140 may include chlorhexidine gluconate. The gel pad 140 may protect the insertion site 25 from micro-organisms and aid in keeping the insertion site 25 clean and disinfected during use of the securement device 100.

The main body 110 may be made of any suitable material. Preferably, the main body 110 may be made of a stretchable material that conforms to undulated surfaces (e.g., the skin 22 of the user 20). The main body 110 may have an elastic modulus from about 0.1 megapascals (MPa) to about 1000 MPa. In some examples, the main body 110 may have an elastic modulus of about 100 MPa, about 200 MPa, about 300 MPa, about 500 MPa, about 700 MPa, about 800 MPa, or about 900 MPa.

The main body 110 may include at least one of polyurethane, neoprene, polyester, butyl rubber, silicone, and polymer foam. Different materials may provide different advantageous characteristics to the main body 110. For example, polymer foam may provide good breathability to the main body 110, while polyurethane may be economical with a good ability to integrate with other materials. Thus, the material of the main body 110 may be selected based on desired application attributes.

The main body 110 may have any suitable shape, such as rectangular, oval, oblong, round, polygonal, and the like. For example, as shown in FIG. 1, the main body 110 may be substantially rectangular with rounded corners. Specifically, the main body 110 may have a maximum length 110L and a maximum width 110W. The maximum length 110L may be defined substantially along the y-axis, while the maximum width 110W may be defined substantially along the x-axis. In one example, the maximum length 110L of the main body 110 may be about 50 millimeters (mm), and the maximum width 110W of the main body 110 may be about 40 mm. Such dimensions of the main body 110 may enable the securement device 100 to be suitable for application at a variety of locations on the user 20.

The securement device 100 further includes a clip 120 coupled to the main body 110 and extending from the first major surface 112. The clip 120 may be coupled to the main body 110 by any suitable method or technique. For example, the clip 120 may be coupled to the main body 110 by molding, heat welding, adhesive coupling, tacking, and the like. The clip 120 is configured to at least partially receive the medical article 10 therethrough and removably secure the medical article 10 to the main body 110. The clip 120 may be adjustable to accommodate medical articles having different shapes and dimensions. The clip 120 will be described in more detail later.

As shown in FIG. 2, the securement device 100 further includes a plurality of microhooks 150 coupled to the main body 110 and extending from the second major surface 114. The plurality of microhooks 150 may be coupled to the main body 110 by any suitable method or technique. For example, the plurality of microhooks 150 may be coupled to the main body 110 by molding, heat welding, adhesive coupling, tacking, and the like. The plurality of microhooks 150 are spaced apart from each other and configured to be at least partially inserted into the skin 22 of the user 20 to secure the main body 110 to the skin 22.

The plurality of microhooks 150 may be made of any material that can penetrate the skin 22 of the user 20. Each of the plurality of microhooks 150 may include at least one of stainless steel, carbon-fiber-reinforced polymer, poly(methyl methacrylate), dissolvable polyester, nickel alloy, and titanium alloy. Each of the plurality of microhooks 150 may further include magnesium alloy. Different materials may provide different advantageous characteristics to the plurality of microhooks 150. For example, stainless steel, nickel alloy, and titanium alloy may improve an ability of the plurality of microhooks 150 to pierce the skin 22, while dissolvable polyester may be economical to use.

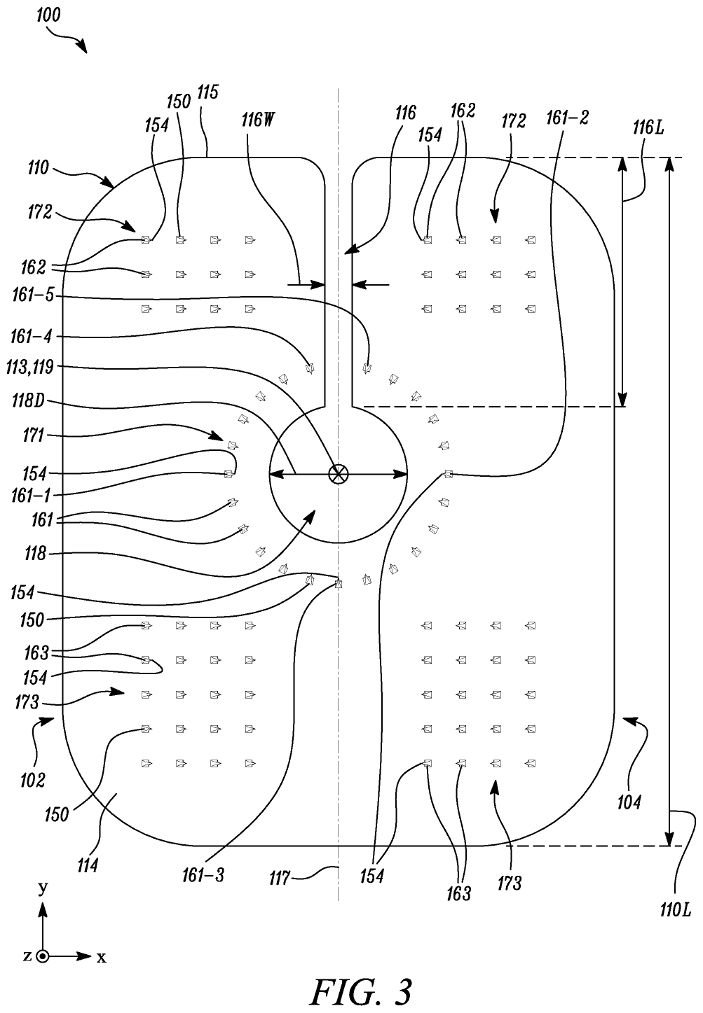

Each microhook 150 from the plurality of microhooks 150 includes a base 152 disposed on the second major surface 114 and a tip 154 distal to the base 152. The plurality of microhooks 150 includes at least one microhook 150-1 and at least one opposing microhook 150-2 facing the at least one microhook 150-1, such that the respective tips 154 of the at least one microhook 150-1 and the at least one opposing microhook 150-2 point towards each other. In other words, an imaginary line extending from the tip 154 of the at least one microhook 150-1 may extend in a general direction of the tip 154 of the at least one opposing microhook 150-2. This directionality of the plurality of microhooks 150 may improve securement of the main body 110 to the skin 22 of the user 20. In the illustrated embodiment of FIG. 2, the plurality of microhooks 150 includes at least one set of microhooks 150 and at least one opposing set of microhooks 150, such that the respective tips 154 of the at least one set of microhooks 150 and the respective tips 154 of the at least one opposing set of microhooks 150 point towards each other. The plurality of microhooks 150 may have many suitable arrangements and configurations. One particular arrangement and configuration of the plurality of microhooks 150 will be described in detail with reference to FIG. 3.

FIG. 3 illustrates a bottom view of the securement device 100 according to an embodiment of the present disclosure.

The second major surface 114 may define a surface centroid 113. The surface centroid 113 may be a geometric center of the second major surface 114. Further, the plurality of microhooks 150 may include at least one first microhook 161. The tip 154 of the at least one first microhook 161 may point towards the surface centroid 113.

In some examples, the at least one first microhook 161 may include a set of first microhooks 161 arranged in a circular array 171 about the surface centroid 113. Further, the tip 154 of each first microhook 161 from the set of first microhooks 161 may point towards the surface centroid 113. In other words, the set of first microhooks 161 may be arranged in the circular array 171 about the surface centroid 113, such that each first microhook 161 points towards the surface centroid 113.

As shown in FIG. 3, the set of first microhooks 161 may include at least two first microhooks 161-1, 161-2 disposed diametrically opposite to each other, such that the respective tips 154 of the at least two first microhooks 161-1, 161-2 point towards each other. The set of first microhooks 161 may further include at least two first microhooks 161-4, 161-5 disposed on opposing sides of the slot 116.

In some examples, the surface centroid 113 of the second major surface 114 may be disposed within the aperture 118. Further, the set of first microhooks 161 may be at least partially arranged around the aperture 118. More specifically, as shown in FIG. 3, the aperture 118 may be circular and may define a center 119. The center 119 of the aperture 118 may coincide with the surface centroid 113 of the second major surface 114. As a result, the set of first microhooks 161 may be at least partially arranged in the circular array 171 around the aperture 118 with the center 119 of the aperture 118 coinciding with a center of the circular array 171.

The set of first microhooks 161 may further include at least one first microhook 161-3 disposed opposite to the slot 116, such that the tip 154 of the at least one first microhook 161-3 points towards the slot 116. In the illustrated embodiment of FIG. 3, the at least one first microhook 161-3 includes only one first microhook 161-3. Since the tip 154 of the one first microhook 161-3 points towards the slot 116, the plurality of microhooks 150 may not include any corresponding microhook whose tip points directly towards or directly opposes the tip 154 of the one first microhook 161-3. In the illustrated embodiment of FIG. 3, apart from the one first microhook 161-3 that is disposed opposite to the slot 116, each first microhook 161 from the set of first microhooks 161 has a corresponding diametrically opposite first microhook 161.

The slot 116 may extend partially along the length of the main body 110. Specifically, the slot 116 may linearly extend from the perimeter 115 towards the surface centroid 113 of the second major surface 114, such that a length 116L of the slot 116 is from about 30% to about 50% of the maximum length 110L of the main body 110. Further, a maximum width 116W of the slot 116 may be less than a diameter 118D of the aperture 118. The diameter 118D being greater than the maximum width 116W may allow the gel pad 140 (shown in FIG. 1) to be received securely within the aperture 118.

The plurality of microhooks 150 may further include a set of second microhooks 162 arranged in a pair of rectangular arrays 172 disposed on opposing sides of the slot 116. One rectangular array 172 may be disposed proximal to the first end 102 of the main body 110, and the other rectangular array 172 may be disposed proximal to the second end 104 of the main body 110.

Further, the tip 154 of each second microhook 162 from the set of second microhooks 162 may point towards the slot 116. In other words, the pair of rectangular arrays 172 may be disposed on opposing sides of the slot 116, such that the tip 154 of each second microhook 162 points towards the slot 116.

Further, each rectangular array 172 includes multiple rows and multiple columns of the second microhooks 162. In the illustrated embodiment of FIG. 3, each row of one rectangular array 172 is substantially aligned with a corresponding row of the other rectangular array 172. In other words, each row of the one rectangular array 172 and the corresponding row of the other rectangular array 172 are arranged substantially along a single line. Moreover, the tips 154 present in each row of the one rectangular array 172 and the tips 154 present in the corresponding row of the other rectangular array 172 point towards each other.

The second major surface 114 may further define at least one line of symmetry 117 (depicted by a dashed line in FIG. 3). Further, the plurality of microhooks 150 further includes a set of third microhooks 163 arranged in a pair of rectangular arrays 173 disposed on opposing sides of the line of symmetry 117. The set of third microhooks 163 may be disposed opposite to the set of second microhooks 162 relative to the aperture 118. The tip 154 of each third microhook 163 from the set of third microhooks 163 may point towards the line of symmetry 117. In other words, the pair of rectangular arrays 173 may be disposed on opposing sides of the line of symmetry 117, such that the tip of each third microhook 163 points towards the line of symmetry 117.

Further, each rectangular array 173 includes multiple rows and multiple columns of the third microhooks 163. In the illustrated embodiment of FIG. 3, each row of one rectangular array 173 is substantially aligned with a corresponding row of the other rectangular array 173. In other words, each row of one rectangular array 173 and the corresponding row of the other rectangular array 173 are arranged substantially along a single line. Moreover, the tips 154 present in each row of the one rectangular array 173 and the tips 154 present in the corresponding row of the other rectangular array 173 point towards each other.

The plurality of microhooks 150 are therefore arranged in the circular array 171 around the surface centroid 113 of the second major surface 114, the pair of rectangular arrays 172 disposed on opposing sides of the slot 116, and the pair of rectangular arrays 173 disposed on opposing sides of the line of symmetry 117 and spaced apart from the pair of rectangular arrays 172. Therefore, the circular array 171 is disposed proximal to a geometric center of the main body 110, the pair of rectangular arrays 172 are disposed proximal to the respective rounded corners of the main body 110 near the slot 116, and the pair of rectangular arrays 173 are disposed proximal to the respective rounded corners of the main body 110 distal to the slot 116. Such an arrangement of the plurality of microhooks 150 may improve attachment between the main body 110 and the skin 22 of the user 20, while reducing an overall density of the plurality of microhooks 150 (i.e., a number of microhooks per unit area) on the second major surface 114.

Having the microhooks 150 pointing towards each other and towards the aperture 118 may also tend to compress the skin towards the line of symmetry 117 and towards the surface centroid 113 where a needle for an IV may be located and inserted through the skin compromising the skin's integrity. By compressing the compromised skin, it is less likely to tear; especially, at the wound created by the needle. Since the main body 110 may be elastic or stretchable in some embodiments, once the microhooks are engaged into the skin after slightly stretching the securement device 100 during application as will be discussed herein later, the elastic main body 110 retracts and the skin may be placed slightly into compression from engagement of the microhooks into the skin.

The plurality of microhooks 150 may be strategically disposed on the second major surface 114, such that upon insertion of the plurality of microhooks 150 into the skin 22 of the user 20, the main body 110 is reliably secured to the skin 22 (shown in FIG. 1) of the user 20 without being uncomfortable or inconvenient to the user 20. To this end, in some examples, at least 50% of an area of the second major surface 114 may be devoid of the plurality of microhooks 150. In some examples, at least 70% of the area of the second major surface 114 may be devoid of the plurality of microhooks 150. In some examples, at least 90% of the area of the second major surface 114 may be devoid of the plurality of microhooks 150. The one or more regions of the second major surface 114 that are devoid of the plurality of microhooks 150 may also facilitate gripping and removal of the main body 110 from the skin 22 without causing discomfort or pain to the user 20.

The arrangement of the plurality of microhooks 150 on the second major surface 114, as shown in FIG. 3, is exemplary in nature, and the arrangement may vary based on desired application attributes.

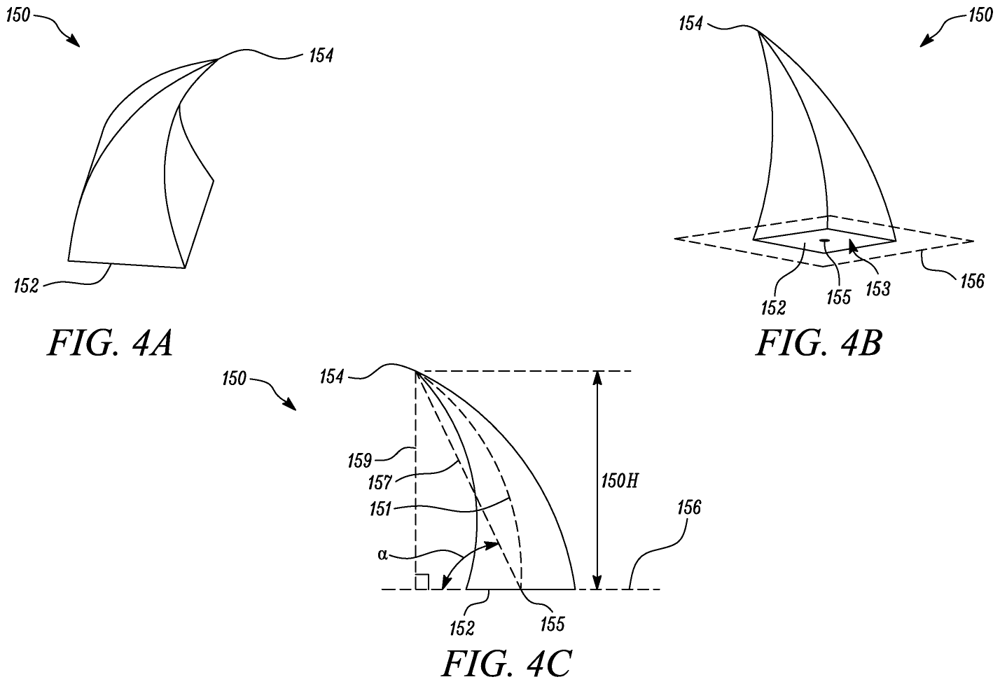

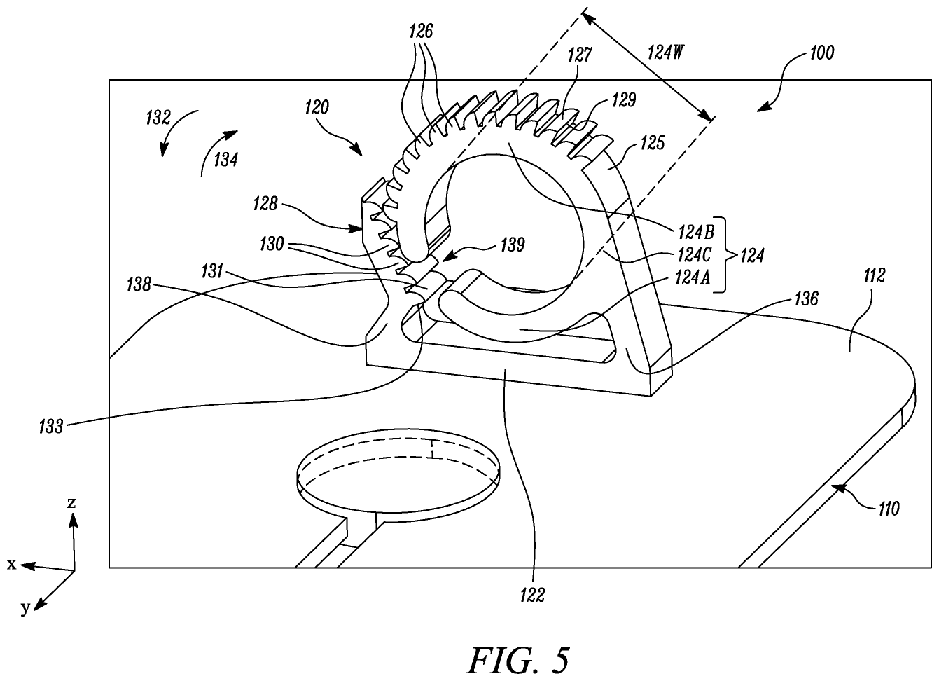

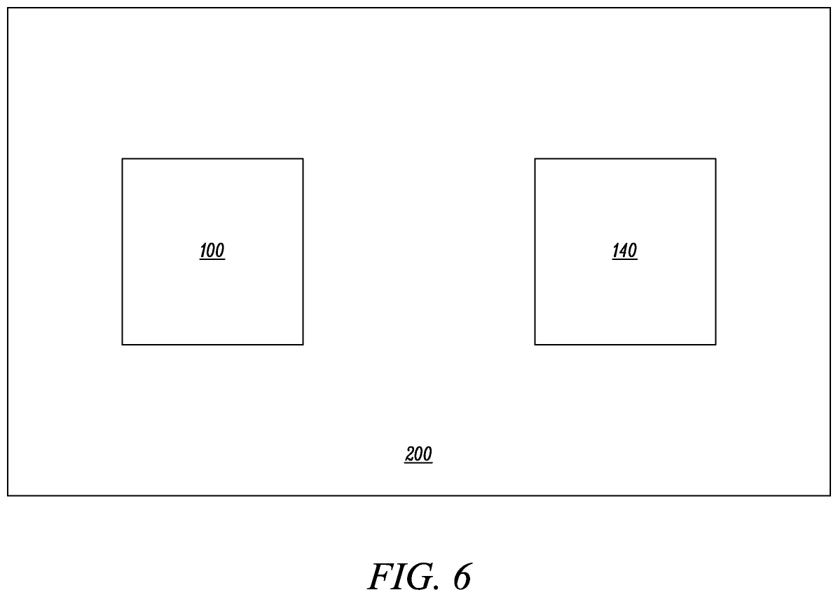

FIGS. 4A-4C schematically illustrate one microhook 150 from the plurality of microhooks 150 according to an embodiment of the present disclosure.

As discussed above, each microhook 150 from the plurality of microhooks 150 includes the base 152 and the tip 154.

Each microhook 150 may define a height 150H between the base 152 and the tip 154. The height 150H may be from about 300 microns (μm) to about 600 μm. As a result, the plurality of microhooks 150 may not penetrate a dermis layer of the skin 22 (shown in FIG. 1) of the user 20. Therefore, insertion of the plurality of microhooks 150 into the skin 22 may not cause pain to the user 20.

As shown in FIG. 4B, at least one microhook 150 from the plurality of microhooks 150 may have a square cross-sectional shape. The at least one microhook 150 may have a pyramidal shape. Furthermore, each microhook 150 from the plurality of microhooks 150 may have a solid configuration devoid of channels. In other words, each microhook 150 may not be hollow.

The base 152 may define a base plane 156 (depicted by a dashed rectangle in FIG. 4B), a base area 153 in the base plane 156, and a base centroid 155 (depicted by a circle in FIG. 4B) of the base area 153. The base area 153 corresponds to an effective area of the base 152 within the base plane 156. The base centroid 155 may correspond to a geometric center of the base area 153 in the base plane 156. Each microhook 150 may further define a hook axis 151 (shown by a dashed line in FIG. 4C) extending from the base centroid 155 to the tip 154 along a length thereof. Each microhook 150 extends between the base 152 and the tip 154 along the hook axis 151. The hook axis 151 may be obtained by joining the centroids of the cross-sectional areas of the microhook 150 from the base 152 to the tip 154. The hook axis 151 may be curvilinear or linear. As shown in FIG. 4C, the hook axis 151 may be curved.

In some examples, an imaginary line 157 (shown by a dashed line in FIG. 4C) joining the tip 154 and the base centroid 155 may be inclined obliquely to the base plane 156. Specifically, as shown in FIG. 4C, the imaginary line 157 may be inclined obliquely to the base plane 156 by an inclination angle α. The inclination angle a may be from about 20 degrees to about 70 degrees. Further, the tip 154 of each microhook 150 may point away from the base 152, such that an imaginary vertical line 159 (shown by a dashed line in FIG. 4C) extending from the tip 154 normal to the base plane 156 lies outside the base 152. This configuration of the tip 154 relative to the base 152 may enable the tip 154 of each microhook 150 to point towards the tip 154 of an opposing microhook 150, the at least one line of symmetry 117 (shown in FIG. 3) of the second major surface 114, and/or the surface centroid 113 (shown in FIG. 3) of the second major surface 114. In various embodiments, the overall shape of the microhooks can resemble a bird of prey's beak or talons with a curving pyramidal structure that ends in a sharp point and, as such, have good penetration into the skin of a patient using the securement device.

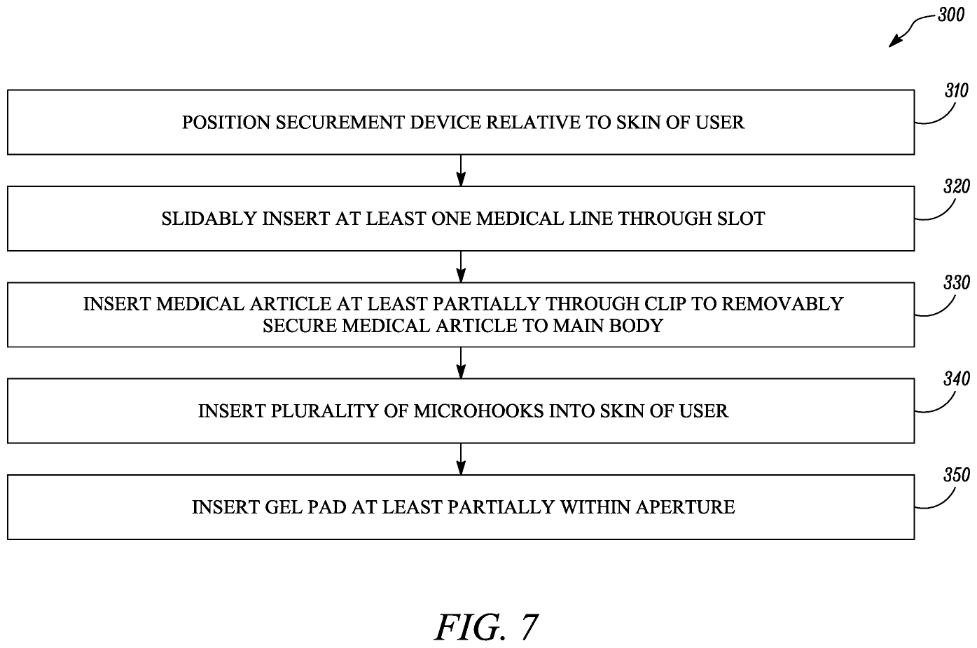

FIG. 5 illustrates a top perspective view of a portion of the securement device 100 according to an embodiment of the present disclosure.

As discussed above, the clip 120 is coupled to the main body 110 and extends from the first major surface 112. Further, the clip 120 is configured to at least partially receive the medical article 10 (shown in FIG. 1) therethrough and removably secure the medical article 10 to the main body 110.

As shown in FIG. 5, the clip 120 may include a clip base 122 coupled to the main body 110 and extending from the first major surface 112 of the main body 110.

The clip 120 may further include a split ring 124 resiliently coupled to the clip base 122. The split ring 124 may be resiliently coupled to the clip base 122 by a ring coupling portion 136. Further, the split ring 124 may be configured to at least partially receive the medical article 10 therethrough (as shown in FIG. 1).

The split ring 124 may include a first ring portion 124A proximal to the clip base 122 and a second ring portion 124B distal to the clip base 122. The second ring portion 124B may be integral with the first ring portion 124A. Further, the second ring portion 124B may be connected to the first ring portion 124A by a ring connecting portion 124C.

The second ring portion 124B may include an outer ring surface 125 facing away from the first ring portion 124A. The second ring portion 124B may further include a plurality of locking teeth 126 extending from the outer ring surface 125.

The split ring 124 may be deformable and movable relative to the clip base 122, such that a gap 139 between the first ring portion 124A and the second ring portion 124B can be adjusted. Adjustment of the gap 139 between the first ring portion 124A and the second ring portion 124B may allow adjustment of a width 124W of the split ring 124. The gap 139 may also allow for the insertion of a medical line to a circular aperture portion of the clip 120 when the second ring portion 124B is not engaged with a locking arm 128. Thereafter, the second ring portion 124B can engage with the locking arm 128 to secure the medical line within the circular aperture portion as best seen in FIG. 1.

The clip 120 may further include the locking arm 128 resiliently coupled to and extending from the clip base 122. Specifically, the locking arm 128 may be resiliently coupled to the clip base 122 by an arm coupling portion 138. The locking arm 128 may include a plurality of detents 130 corresponding to a plurality of predetermined widths of the split ring 124.

The second ring portion 124B may be resiliently movable relative to the locking arm 128 in a first direction 132. Each detent 130 from the plurality of detents 130 may be configured to selectively and lockingly engage with a corresponding locking tooth 126 from the plurality of locking teeth 126 in order to prevent the second ring portion 124B from moving in a second direction 134 opposite to the first direction 132. Therefore, the plurality of detents 130 and the plurality of locking teeth 126 may form a ratchet and pawl arrangement.

Specifically, as shown in FIG. 5, each locking tooth 126 may include a tooth curved surface 127 and a tooth planar surface 129 opposite to the tooth curved surface 127. Further, each detent 130 may include a detent curved surface 131 and a detent planar surface 133. The tooth curved surface 127 may be configured to movably engage with the detent curved surface 131 to allow movement of the second ring portion 124B in the first direction 132. Further, the tooth planar surface 129 may be configured to lockingly engage with the detent planar surface 133 to prevent the second ring portion 124B from moving in the second direction 134.

Therefore, the width 124W of the split ring 124 may be adjusted by selectively lockingly engaging one or more of the plurality of detents 130 with corresponding one or more of the plurality of locking teeth 126, such that the medical article 10 is at least partially receivable therethrough (as shown in FIG. 1). The second ring portion 124B may be moved towards the clip base 122 along the first direction 132 in order to decrease the gap 139 and the width 124W. When a desired width 124W is achieved, releasing the second ring portion 124B may cause engagement of the detent planar surfaces 133 of one or more of the plurality of detents 130 with corresponding tooth planar surfaces 129 of one or more of the plurality of locking teeth 126, such that the split ring 124 is secured at the desired width 124W. In order to increase the width 124W, for example, for removal of the medical article 10, the locking arm 128 and the split ring 124 may be moved away from each other, such that the plurality of detents 130 may disengage from the plurality of locking teeth 126. As a result, the second ring portion 124B may be free to move in the second direction 134, thereby allowing a desired increase in the gap 139 and the width 124W or the second ring portion to 124B to completely disengage from the locking arm 128 if desired to remove the medical line from the circular aperture portion.

The split ring 124 may be designed to accommodate a variety of medical articles. For example, the width 124W of the split ring 124 may be adjustable between about 1 mm and 3 mm. This may enable the split ring 124 to receive medical articles of standardized shapes and dimensions.

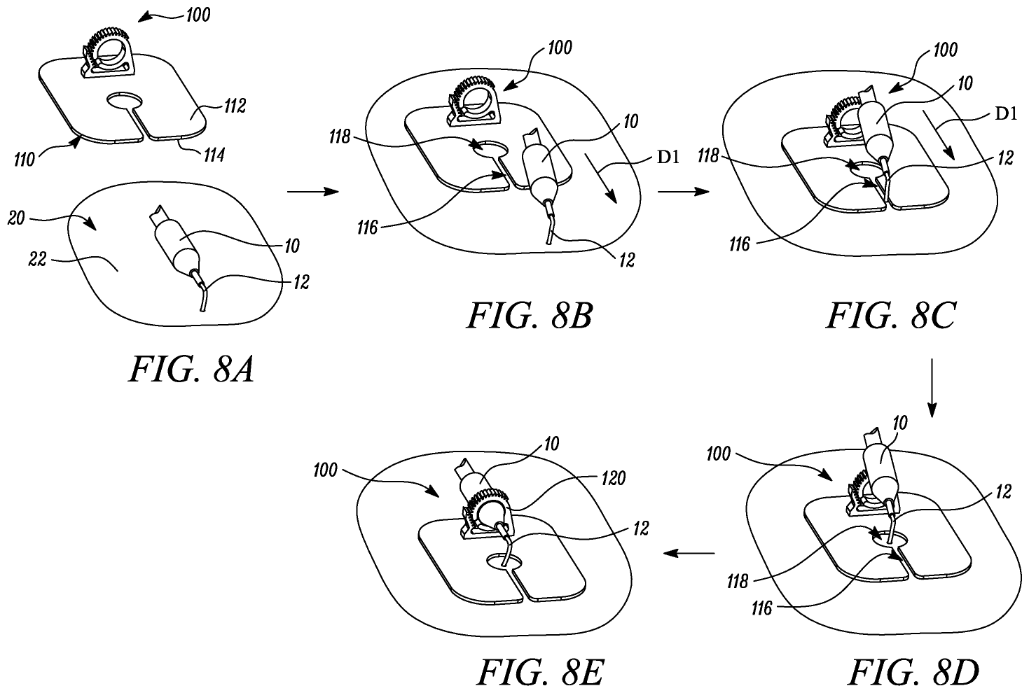

FIG. 6 illustrates a schematic block diagram of a kit 200 according to an embodiment of the present disclosure.

The kit 200 includes the securement device 100 and the gel pad 140. As discussed above, the gel pad 140 may be configured to be at least partially received within the aperture 118 (shown in FIG. 1) and to cover the insertion site 25. The kit 200 may further include gloves, a sterilizing material, a cloth or other absorbent material, and an antiseptic agent (e.g., chlorhexidine gluconate). The kit 200 may further include cleaning articles, such as cleaning cloth, cotton balls, cotton swabs, and the like. The kit 200 may be available to a clinician for use in a sterilized package.

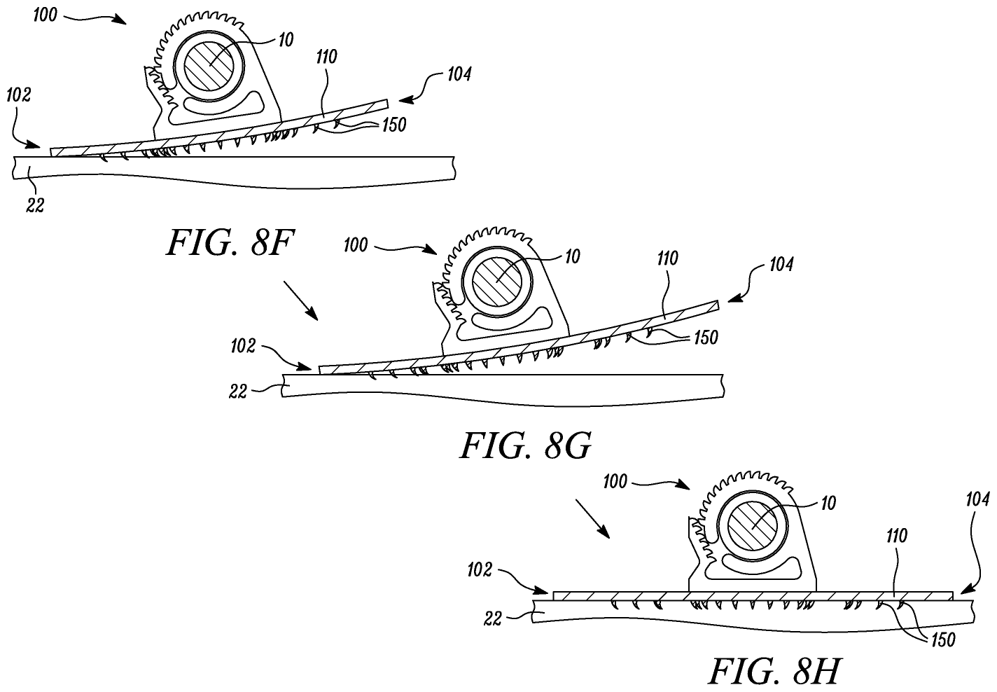

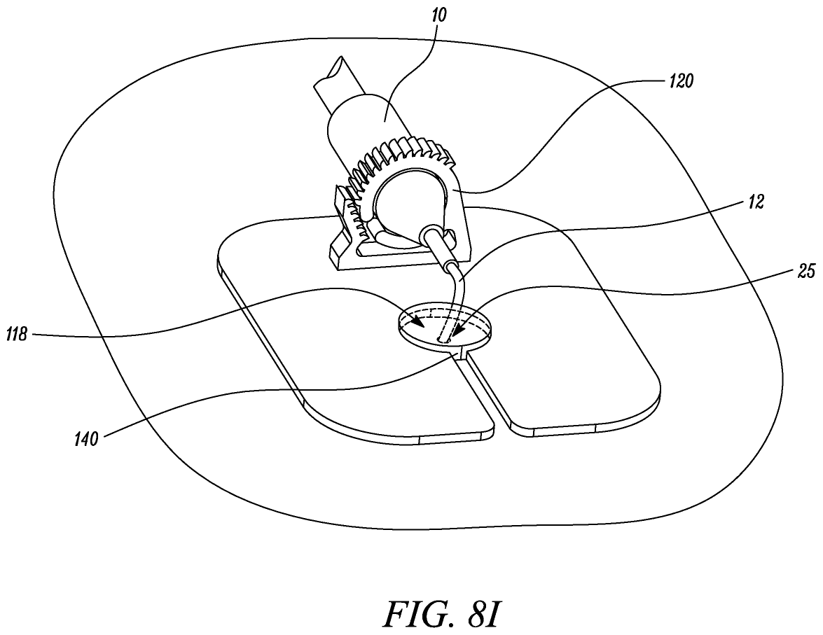

FIG. 7 illustrates a flowchart depicting a method 300 of using the kit 200 of FIG. 6 according to an embodiment of the present disclosure. The method 300 is also schematically illustrated in FIGS. 8A-8I, and will be discussed with reference to FIGS. 7 and 8A-8I.

Referring to FIG. 8A, at step 310, the method 300 includes positioning the securement device 100 relative to the skin 22 of the user 20, such that the second major surface 114 of the main body 110 faces the skin 22.

At step 320, the method 300 further includes slidably inserting the at least one medical line 12 through the slot 116, such that the at least one medical line 12 is at least partially received through the aperture 118 of the main body 110. For example, as shown in FIGS. 8B-8D, the securement device 100 may be slid along a direction D1 towards the at least one medical line 12, such that the at least one medical line 12 is slidably inserted through the slot 116, and the at least one medical line 12 is at least partially received through the aperture 118 of the main body 110.

Referring to FIG. 8E, at step 330, the method 300 further includes inserting the medical article 10 at least partially through the clip 120 to removably secure the medical article 10 to the main body 110. In some examples inserting the medical article at least partially through the clip 120 may occur when there is a gap when the second ring portion 124B is not engaged with the locking arm 128. In some examples, inserting the medical article 10 at least partially through the clip 120 may further include adjusting a width of the clip 120 (i.e., the width 124W of the split ring 124 as shown in FIG. 5 and discussed above) based on a width of the medical article 10.

At step 340, the method 300 further includes inserting the plurality of microhooks 150 into the skin 22 of the user 20, such that the main body 110 is secured to the skin 22 of the user 20.

In some examples, inserting the plurality of microhooks 150 into the skin 22 of the user 20 may further include inserting at least some of the plurality of microhooks 150 proximal to the first end 102 of the main body 110 into the skin 22 of the user 20; stretching the main body 110; and inserting at least some of the plurality of microhooks 150 proximal to the second end 104 of the main body 110 opposing the first end 102 into the skin 22 of the user 20. For example, as shown in FIG. 8F-8H, after insertion of at least some of the plurality of microhooks 150 proximal to the first end 102 of the main body 110 into the skin 22, the main body 110 may be stretched at the second end 104, and subsequently, at least some of the plurality of microhooks 150 proximal to the second end 104 of the main body 110 may be inserted into the skin 22.

Referring to FIG. 8I, at step 350, the method 300 further includes inserting the gel pad 140 at least partially within the aperture 118, such that the gel pad 140 covers the insertion site 25. The gel pad 140 may include pre-made slits in order to receive the at least one medical line 12 therethrough.

The method 300 may allow a clinician or a user to conveniently use the kit 200, and secure the medical article 10 to the skin 22 via the securement device 100.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A securement device for securing a medical article having at least one medical line insertable into a skin of a user at an insertion site, the securement device comprising:
a main body comprising:
a first major surface;
a second major surface opposite to the first major surface, wherein the second major surface is configured to face the skin of the user;
a perimeter extending between the first major surface and the second major surface; and
a slot extending from the perimeter and extending through the main body from the first major surface to the second major surface, wherein the slot is configured to at least partially and slidably receive the at least one medical line therethrough;
a clip coupled to the main body and extending from the first major surface, wherein the clip is configured to at least partially receive the medical article therethrough and removably secure the medical article to the main body; and
a plurality of microhooks coupled to the main body and extending from the second major surface, wherein the plurality of microhooks are spaced apart from each other and configured to be at least partially inserted into the skin of the user to secure the main body to the skin, wherein each microhook from the plurality of microhooks comprises a base disposed on the second major surface and a tip distal to the base, wherein the plurality of microhooks comprises at least one microhook and at least one opposing microhook facing the at least one microhook, such that the respective tips of the at least one microhook and the at least one opposing microhook point towards each other.

2. The securement device of claim 1, wherein the second major surface defines a surface centroid, wherein the plurality of microhooks comprises at least one first microhook, and wherein the tip of the at least one first microhook points towards the surface centroid.

3. The securement device of claim 2, wherein the at least one first microhook comprises a set of first microhooks arranged in a circular array about the surface centroid, wherein the tip of each first microhook from the set of first microhooks points towards the surface centroid.

4. The securement device of claim 3, wherein the main body further comprises an aperture extending therethrough and communicating with the slot, wherein the aperture is configured to at least partially receive the at least one medical line therethrough, wherein the aperture is further configured to at least partially receive a gel pad therein, wherein the surface centroid of the second major surface is disposed within the aperture, and wherein the set of first microhooks are at least partially arranged around the aperture.

5. The securement device of claim 4, wherein the aperture is circular and defines a center, and wherein the center of the aperture coincides with the surface centroid of the second major surface.

6. The securement device of claim 5, wherein a maximum width of the slot is less than a diameter of the aperture.

7. The securement device of claim 3, wherein the set of first microhooks comprises at least two first microhooks disposed diametrically opposite to each other, such that the respective tips of the at least two first microhooks point towards each other.

8. The securement device of claim 3, wherein the set of first microhooks comprises at least one first microhook disposed opposite to the slot, such that the tip of the at least one first microhook points towards the slot.

9. The securement device of claim 3, wherein the set of first microhooks comprises at least two first microhooks disposed on opposing sides of the slot.

10. The securement device of claim 2, wherein the slot linearly extends from the perimeter towards the surface centroid of the second major surface, such that a length of the slot is from about 30% to about 50% of a maximum length of the main body.

11. The securement device of claim 1, wherein the plurality of microhooks comprises a set of second microhooks arranged in a pair of rectangular arrays disposed on opposing sides of the slot, and wherein the tip of each second microhook from the set of second microhooks points towards the slot.

12. The securement device of claim 1, wherein the second major surface defines at least one line of symmetry, wherein the plurality of microhooks comprises a set of third microhooks arranged in a pair of rectangular arrays disposed on opposing sides of the line of symmetry, wherein the tip of each third microhook from the set of third microhooks points towards the line of symmetry.

13. The securement device of claim 1, wherein at least 50% of an area of the second major surface is devoid of the plurality of microhooks.

14. The securement device of claim 1, wherein the base defines a base plane, a base area in the base plane, and a base centroid of the base area, and wherein an imaginary line joining the tip and the base centroid is inclined obliquely to the base plane.

15. The securement device of claim 14, wherein the imaginary line is inclined obliquely to the base plane by an inclination angle from about 20 degrees to about 70 degrees.

16. The securement device of claim 14, wherein each microhook defines a hook axis extending from the base centroid to the tip along a length thereof, and wherein the hook axis is curvilinear or linear.

17. The securement device of claim 14, wherein the tip of each microhook points away from the base, such that an imaginary vertical line extending from the tip normal to the base plane lies outside the base.

18. The securement device of claim 1, wherein at least one microhook from the plurality of microhooks has a square cross-sectional shape.

19. The securement device of claim 1, wherein each microhook from the plurality of microhooks defines a height between the base and the tip, and wherein the height is from about 300 microns (μm) to about 600 μm.

20. The securement device of claim 1, wherein each microhook from the plurality of microhooks has a solid configuration devoid of channels.

21. The securement device of claim 1, wherein the main body has an elastic modulus from about 0.1 megapascals (MPa) to about 1000 MPa.

22. The securement device of claim 1, wherein the main body comprises at least one of polyurethane, neoprene, polyester, butyl rubber, silicone, and polymer foam.

23. The securement device of claim 1, wherein each of the plurality of microhooks comprises at least one of stainless steel, carbon-fiber-reinforced polymer, poly(methyl methacrylate), dissolvable polyester, nickel alloy, and titanium alloy.

24. The securement device of claim 1, wherein the main body is substantially rectangular with rounded corners.

25. The securement device of claim 1, wherein the clip comprises:
a clip base coupled to the main body and extending from the first major surface of the main body;
a split ring resiliently coupled to the clip base, the split ring comprising a first ring portion proximal to the clip base, a second ring portion distal to the clip base and comprising an outer ring surface facing away from the first ring portion, and a plurality of locking teeth extending from the outer ring surface, and wherein the split ring is configured to at least partially receive the medical article therethrough; and
a locking arm resiliently coupled to and extending from the clip base, wherein the locking arm comprising a plurality of detents corresponding to a plurality of predetermined widths of the split ring;
wherein the second ring portion is resiliently movable relative to the locking arm in a first direction, and wherein each detent from the plurality of detents is configured to selectively and lockingly engage with a corresponding locking tooth from the plurality of locking teeth in order to prevent the second ring portion from moving in a second direction opposite to the first direction.

26. The securement device of claim 25, wherein each locking tooth comprises a tooth curved surface and a tooth planar surface opposite to the tooth curved surface, wherein each detent comprises a detent curved surface and a detent planar surface, wherein the tooth curved surface is configured to movably engage with the detent curved surface to allow movement of the second ring portion in the first direction, and wherein the tooth planar surface is configured to lockingly engage with the detent planar surface to prevent the second ring portion to move in the second direction.

27-55. (canceled)
